(19) United States
(12) Patent Application Publication
(10) Pub. No.: US 2017/0360932 A1
(43) Pub. Date: Dec. 21, 2017

(54) IMMUNE MODULATORS IN COMBINATION WITH RADIATION TREATMENT

(71) Applicant: Varian Medical Systems, Inc., Palo Alto, CA (US)

(72) Inventor: Renate Parry, Oakland, CA (US)

(73) Assignee: Varian Medical Systems, Inc., Palo Alto, CA (US)

(21) Appl. No.: 15/625,869

(22) Filed: Jun. 16, 2017

Related U.S. Application Data
(60) Provisional application No. 62/351,681, filed on Jun. 17, 2016.

(51) Int. Cl. A61K 41/00; A61N 5/10; C07K 16/28; A61K 39/395; G01N 33/574; C07K 16/18; A61K 39/00

(52) U.S. Cl. CPC A61K 41/0038; G01N 33/57423; A61N 5/10; C07K 16/18; C07K 16/2803; A61K 39/3955; G01N 33/57484; A61K 2039/505; A61N 2005/1098; G01N 2333/495; G01N 2333/70503; G01N 2333/70585; G01N 2800/52

(57) ABSTRACT

Methods for treating tumors by administering ionizing radiation and an immune modulator to a patient with cancer are disclosed. The methods provide the dual benefits of anti-tumor efficacy plus normal tissue protection when combining immune modulators with ionizing radiation to treat cancer patients. The methods described herein also allow for the classification of patients into groups for receiving optimized radiation treatment in combination with an immune modulator based on patient-specific biomarker signatures.

### CROSS-REFERENCE TO RELATED APPLICATIONS

The present application is a non-provisional application of, and claims the benefit and priority under 35 U.S.C. 119(e) of U.S. Provisional Application No. 62/351,681, filed Jun. 17, 2016, entitled “IMMUNE MODULATORS IN COMBINATION WITH RADIATION TREATMENT,” the contents of which is incorporated herein by reference for all purposes.

### BACKGROUND OF THE INVENTION

Radiation therapy is a key therapeutic modality for patients with cancer. Radiation can be delivered to the tumor with submillimeter precision while mostly sparing normal tissue, ultimately leading to tumor cell killing. However, the tumor cell's ability to escape the cell killing effects of radiation and/or to develop resistance mechanisms can counteract the tumor cell killing action of radiotherapy, potentially limiting the therapeutic effect of radiotherapy to treat cancer. Furthermore, the potential for normal tissue toxicity can impact the therapeutic window of radiation therapy as a treatment paradigm.

Radiation-induced tumor cell death leads to release of tumor antigens from lysed cells, increased MHC-1 expression on antigen presenting cells, and enhanced diversity of the intratumoral T-cell population. These factors and others are key to initiate activation of the body's own immune systems to eradicate cancer cells. Immune modulators are being explored to activate the body's own immune system, but are known to have limitations as monotherapy (e.g., response rate in patients). The response rate of immune modulators when used as monotherapy is in the range of 20-30% of the targeted patient population. Combination approaches such as using two immune modulators or an immune modulator with a targeted anti-cancer drug have limitations due to systemic normal tissue toxicity.

### BRIEF SUMMARY OF THE INVENTION

The methods described herein provide the dual benefits of anti-tumor efficacy and normal tissue protection when combining an immune modulator with ionizing radiation to treat cancer patients. Methods described herein can be used to treat local and metastatic cancers by administering ionizing radiation therapy to deliver a highly conformal dose to the tumor, and an immune modulator. This combination therapy has the potential to improve both the efficacy of radiation therapy both locally and systemically, and the efficacy of the immune modulators. The methods described herein also allow for the classification of patients into groups for receiving optimized radiation treatment based on patient specific biomarker signatures. The biomarker signature includes markers that have been shown to correlate with tumor agressiveness, radioresistance and poor prognosis.

In some aspects, provided herein is a method for treating a tumor in a subject with cancer comprising administering ionizing radiation and an immune modulator to the tumor. In some embodiments, the amount of ionizing radiation and immune modulator administered to the subject is effective at treating the tumor, for example, effective at killing one or more tumor cells, reducing the growth rate or size of the tumor, or eliminating the tumor from the body of the subject. In some embodiments, the immune modulator is selected from the group consisting of an inhibitor to an inhibitory checkpoint molecule, an activator of a stimulatory checkpoint molecule, a chemokine inhibitor, an inhibitor of macrophage migration inhibitory factor (MIF), a growth factor, a cytokine, an interleukin, an interferon, an antibody that binds to an immune system cell, a cellular immune modulator, a vaccine, an oncolytic virus, and any combination thereof. Administration of the immune modulator was unexpectedly found to increase the anti-tumor response when combined with radiation therapy.

In some embodiments, the inhibitor to the inhibitory checkpoint molecule is a small molecule drug, or an antibody or a fragment thereof that specifically binds to the inhibitory checkpoint molecule and inhibits its activity, wherein the inhibitory checkpoint molecule is selected from the group consisting of PD-1, PD-L1, PD-L2, CTLA-4, BTLA, A2aR, B7-H2, B7-H3, B7-H4, B7-H6, CD47, CD48, CD160, CD244 (2B4), CHK1, CHK2, CGEN-15049, ILT-2, ILT-4, LAG-3, VISTA, gp49B, PIR-B, TIGIT, TIM1, TIM2, TIM3, TIM4, and KIR, and ligands thereof. In some embodiments, the activator of the stimulatory checkpoint molecule is a small molecule drug, polypeptide-based activator, or polynucleotide-based activator that specifically binds to the stimulatory checkpoint molecule and increases its activity, wherein the stimulatory checkpoint molecule is selected from the group consisting of B7-1 (CD80), B7-2 (CD86), 4-1BB (CD137), OX40 (CD134), HVEM, inducible costimulator (ICOS), glucocorticoid-induced tumor necrosis factor receptor (GITR), CD27, CD28, CD40, and ligands thereof. In some instances, the chemokine inhibitor is a small molecule drug, or antibody or fragment thereof that specifically binds to the chemokine (or its receptor) and inhibits chemokine activity. In some embodiments, the chemokine is selected from the group consisting of CCL2, CCL3, CCL4, CCL5, CCL7, CCL8, CCL11, CCL12, CCL13, CCL14, CCL15, CCL16, CCL17, CCL18, CCL19, CCL20, CCL21, CCL22, CCL23, CCL24, CCL5, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL5, and CXCL16. In some embodiments, the chemokine inhibitor binds to a chemokine receptor selected from the group consisting of CCR1, CCR2, CCR3, CCR, 4, CCR5, CCR6, CCR7, CCR8, CCR9, CCR10, CXCR1, CXCR2, CXCR3, CXCR4, CXCR5, CXCR6, and CXCR7. In some cases, the inhibitor of MIF is a small molecule drug, or antibody or fragment thereof that specifically binds to MIF and inhibits MIF activity.

In some aspects, provided herein is a method for treating a tumor in a subject with cancer comprising administering ionizing radiation and an immune modulator to the tumor. The method comprises (a) determining an expression level of one or more biomarkers in a tumor sample from the subject, wherein the one or more biomarkers are selected from the group consisting of an immune cell marker(s), tumor cell marker(s), circulating marker(s), and any combination thereof; (b) comparing the expression level of the one or more biomarkers to an expression level of the one or more biomarkers in a normal tissue sample; and (c) administering to the tumor in the subject a treatment comprising ionizing radiation and an immune modulator if the expression level of the one or more biomarkers in the tumor sample is modified compared to the expression level in the normal tissue sample. The biomarker can be CD44, milk fat globule-EGF factor 8 (MFG-E8), CD68, TGFβ, a TGFβ-pathway related biomarker, or any combination thereof.

In certain aspects, provided herein is a method of identifying a subject with cancer as a candidate for treatment comprising ionizing radiation and an immune modulator. The method includes: (a) determining an expression level of one or more biomarkers in a tumor sample from the subject, wherein the one or more biomarkers are selected from the group consisting of an immune cell marker(s), tumor cell marker(s), circulating marker(s), imaging marker(s), and any combination thereof; (b) comparing the expression level of the one or more biomarkers to an expression level of the one or more biomarkers in a normal tissue sample; and (c) classifying the subject as a candidate for treatment comprising ionizing radiation and the immune modulator if the expression level of the one or more biomarkers in the tumor sample is modified compared to the expression level in the normal tissue sample. The biomarker can be CD44, MFG-E8, CD68, TGFβ, a TGFβ-pathway related biomarker, or any combination thereof.

In other aspects, provided herein is a method of selecting a treatment for a subject with cancer. The method comprises: (a) determining an expression level of one or more biomarkers in a tumor sample from the subject, wherein the one or more biomarkers are selected from the group consisting of an immune cell marker(s), tumor cell marker(s), circulating marker(s), and any combination thereof; (b) comparing the expression level of the one or more biomarkers to an expression level of the one or more biomarkers in a normal tissue sample; and (c) selecting a treatment comprising ionizing radiation and an immune modulator if the expression level of the one or more biomarkers in the tumor sample is modified compared to the expression level in the normal tissue sample. The biomarker can be CD44, MFG-E8, CD68, TGFβ, a TGFβ-pathway related biomarker, or any combination thereof.

In some embodiments, the subject is administered ionizing radiation and/or combination therapy comprising ionizing radiation and an immune modulator if the expression level of CD44 is increased and/or the expression level of MFG-E8 is decreased relative to the expression level in a normal or control sample. In some embodiments, the amount of ionizing radiation and/or the amount of an immune modulator administered to the subject is increased if the expression level of CD44 is increased and/or the expression level of MFG-E8 is decreased relative to the expression level in a normal or control sample. On the other hand, the amount of ionizing radiation and/or the amount of an immune modulator administered to the subject can be decreased if the expression level of CD44 is decreased and/or the expression level of MFG-E8 is increased relative to the expression level in a normal or control tissue sample.

In some embodiments, the subject is administered ionizing radiation and/or combination therapy comprising ionizing radiation and an immune modulator if the expression level of CD68 is increased relative to the expression level in a normal or control tissue sample. In some embodiments, the amount of ionizing radiation and/or the amount of an immune modulator administered to the subject is increased if the expression level of CD68 is increased relative to the expression level in a normal or control tissue sample. On the other hand, the amount of ionizing radiation and/or the amount of an immune modulator administered to the subject can be decreased if the expression level of CD68 is decreased relative to the expression level in a normal or control tissue sample.

Provided herein are improved methods for treating a tumor that include administering an immune modulator and ionizing radiation to the subject with cancer. This combination therapy can elicit an increased anti-cancer response compared to immune modulator monotherapy or radiation monotherapy.

In some aspects, provided herein is use of ionizing radiation and an immune modulator for treating a tumor in a subject. In some embodiments, the use comprises a combination of ionizing radiation and an immune modulator described herein.

In another aspect, the disclosure provides an immune modulator for use in a method of treating a tumor in a subject with cancer, characterized in that the method comprises administering ionizing radiation and the immune modulator to the tumor.

In another aspect, provided herein is an immune modulator for use in a method of treating a tumor in a subject with cancer, characterized in that the method comprises:

(a) determining an expression level of one or more biomarkers in a tumor sample from the subject, wherein the one or more biomarkers are selected from the group consisting of an immune cell marker(s), tumor cell marker(s), circulating marker(s), and any combination thereof;

(b) comparing the expression level of the one or more biomarkers to an expression level of the one or more biomarkers in a normal tissue sample; and

(c) administering to the tumor in the subject a treatment comprising ionizing radiation and an immune modulator if the expression level of the one or more biomarkers in the tumor sample is modified compared to the expression level in the normal tissue sample.

Other objects, features, and advantages of the present invention will be apparent to one of skill in the art from the following detailed description and figures.

### BRIEF DESCRIPTION OF THE DRAWINGS

FIG. 1: Patient cohort treated at HFHS with either stereotactic body radiation therapy (SBRT) (12 Gy×4) or conventional fractionated radiation (60 to 70 Gy).

FIG. 2 shows the role of CD44 and CD44-related signaling pathways (TGFβ pathway) in cancer. Modified from Thapa R, Wilson, G D: Stem cells Int, (2016).

FIG. 3A shows Allred IHC scoring, taking into account intensity and proportion of protein expression in cells. FIG. 3B shows expression levels of CD44 and MGF-E8 in lung tumor tissues.

FIG. 4 illustrates the role of TGFβ during radiation treatment.

FIGS. 5A, 5B, and 5C show that TGFβ activity in human NSCL histological subtypes correlates with radiation resistance. Immunostaining of ACD and SCC tumor samples is shown in FIG. 5A. FIGS. 5B and 5C compare the level of TGFβ and activated SMAD2 in ACD and SCC samples.

FIG. 6 shows that combination treatment comprising an immune modulator and radiation can enhance inhibition of tumor growth compared to monotherapy.

FIG. 7 shows that combination treatment comprising an immune modulator and radiation can enhance inhibition of tumor growth compared to monotherapy.

FIGS. 8A-8E show TIM-4 expression in human lung tumor (FIG. 8A), colon tumor (FIG. 8B), prostate tumor (FIG. 8C) and breast tumor (FIG. 8D) and in a colon tumor bearing syngeneic C57/BL6 mouse model (FIG. 8E). FIG. 8F is the negative control.

FIGS. 9A-9D show MFGE-8 expressin in human lung tumor (FIG. 9A), human colon tumor (FIG. 9B), human prostate tumor (FIG. 9C), and human breast tumor (FIG. 9D).

FIGS. 10A and 10B show that treatment comprising an immune modulator (anti-TIM-4 antibody) in combination with radiation can inhibit tumor growth compared to monotherapy with the immune modulator. FIG. 10A shows MC-38 carcinoma bearing mice were treated with anti-TIM4 antibody (2 mg/kg) on days 17.19,21,23. Tumor volumes of individual mice (C1-C5) were monitored over the course of the treatment. FIG. 10B shows MC-38 carcinoma bearing mice were treated with radiation (2 Gy) at day 16, followed by anti-TIM4 antibody administration (2 mg/kg) on days 17.19,21,23. Tumor volumes of individual mice (D1-D5) wer monitored over the course of the treatment.

### DETAILED DESCRIPTION OF THE INVENTION

The methods described herein provide the advantages of anti-tumor efficacy and normal tissue protection when combining an immune modulator with ionizing radiation to treat cancer patients. The methods described herein provide the unexpected result that ionizing radiation in combination with immune modulator therapy can increase the anti-tumor response compared to treatment with radiation therapy or immune modulator therapy alone (monotherapy). The increase in the anti-tumor response can enhance or increase the inhibition of tumor growth that is provided by either monotherapy alone. Methods described herein can be used to treat local and metastatic cancers by administering ionizing radiation therapy to deliver a highly conformal dose to the tumor, and an immune modulator. The combination therapy described herein can improve both the efficacy of radiation therapy (locally and systemically) and the efficacy of the immune modulators. The immune modulator also enhances the anti-cancer response when administered in combination with radiation, compared to administration of either an immune modulator alone or radiation monotherapy.

### I. DEFINITIONS

The term “treating” refers to administering a treatment to a tumor or the subject diagnosed with a tumor. The treatment can be administered in an amount or therapeutic dose that is sufficient or effective to kill tumor cells, slow the growth of the tumor, reduce the size of the tumor, or eliminate the tumor from the subject entirely. Examples of treatments include ionizing radiation, an immune modulator agent, or a combination of both. The term also includes selecting a treatment or treatment plan, and providing treatment options to a healthcare provider or the subject.

The term “ionizing radiation” refers to radiation comprising particles having enough kinetic energy to discharge an electron from an atom or molecule, thereby producing an ion. The term includes both directly ionizing radiation, such as that caused by atomic particles such as alpha particles (helium nuclei), beta particles (electrons), and protons, and indirectly ionizing radiation, such as photons, including gamma rays and x-rays. Examples of ionizing radiation used in radiation therapy include high energy x-rays, electron beams, and proton beams.

The term “tumor environment” or “tumor micro-environment” refers to the immediate small-scale environment of an organism or part of an organism, especially as a distinct part of a larger environment, for example, the immediate small-scale environment of the tumor. The term includes not only the tumor cells themselves, but associated blood-vessels (including endothelial cells and smooth muscle cells), immune system cells and secreted cytokines, epithelial cells, fibroblasts, connective tissue, and/or extracellular matrix that is associated with or surrounds the tumor. The term also refers to the cellular and extracellular environment in which the tumor is located.

The term “standard of care” or “standard radiation treatment protocol” in radiation therapy generally refers to the ionizing radiation dose and administration interval that is generally accepted in the medical field as appropriate treatment for a given tumor, based on the tumor type, size, tissue location, and various other biological parameters. The standard of care or standard treatment protocol varies and is dependent on several factors. For example, for radiation therapy of lung cancer, the standard of care includes multiple fractions (e.g., approximately 30 fractions of low dose radiation, or approximately 60 Gy over 6 weeks) or a smaller number of fractions (e.g., 1-5 fractions) of biologically active doses (e.g., 54 GY in 3 fractions for peripheral tumors, or 48-60 Gy in 4-8 fractions for central tumors) administered to the tumor.

The term “similar dose of ionizing radiation” refers to a dose of ionizing radiation that is identical to, nearly the same, or substantially the same as the effective dose administered to a tumor in another subject, or administered to a tumor in the same subject undergoing an existing course of treatment. The term encompasses the normal and expected variation in ionizing radiation doses delivered by a medical technician skilled in the art of administering ionizing radiation to a tumor in a subject. For example, the term encompasses variation in the effective dose administered to a tumor of less than 10%, less than 5%, or less than 1%. The subject can be a human or non-human animal, such as a companion animal (e.g., cat, dog) or farm animal (e.g., cow, horse, etc.).

The term “expression level” refers to the amount or level and/or the presence or absence of a biomarker described herein.

The term “small molecule drug” refers to an organic compound having a molecular weight of less than about 50 kDa, less than about 10 kDa, less than about 1 kDa, less than about 900 daltons, or less than about 500 daltons. The term includes drugs having desired pharmacological properties, and includes compounds that can be administered orally or by injection.

The term “radiosensitizer” refers to any substance that makes tumor cells easier to kill with radiation therapy. Exemplary radiosensitizers include hypoxia radiosensitizers such as misonidazole, metronidazole, and trans-sodium crocetinate, and DNA damage response inhibitors such as Poly (ADP) ribose polymerase (PARP) inhibitors.

The terms “sample,” “biological sample,” and “tumor sample” refer to bodily fluid, such as but not limited to blood, serum, plasma, or urine, and/or cells or tissues obtained from a subject or patient. In some embodiments, the sample is a formalin-fixed and paraffin embedded tissue or tumor sample. In some embodiments, the sample is a frozen tissue or tumor sample. In some embodiments, the tumor sample can be a biopsy comprising tumor cells from the tumor.

### II. DETAILED DESCRIPTION OF THE EMBODIMENTS

The present disclosure describes methods for treating a tumor in a subject by determining the expression levels of signature biomarkers in a tumor sample, comparing the expression levels in the tumor sample to the expression levels in a normal tissue sample, and treating the tumor if the expression levels in the tumor sample are different from those in the normal tissue sample. In some embodiments, the treatment is ionizing radiation in combination with one or more immune modulators. Thus, the biomarkers provide so called “companion diagnostics” for the therapy to treat tumors. Methods described herein can be used to treat local and metastatic cancers by administering ionizing radiation therapy to deliver a highly conformal dose to the tumor, and an immune modulator.

In one aspect, a method for treating a tumor in a subject with cancer comprising administering ionizing radiation and an immune modulator to the tumor is provided. The immune modulator can be selected from the group consisting of an inhibitor to an inhibitory checkpoint molecule, an activator of a stimulatory checkpoint molecule, a chemokine inhibitor, an inhibitor of macrophage migration inhibitory factor (MIF), a growth factor, a cytokine, an interleukin, an interferon, an antibody that binds to an immune system cell, such as a bispecific antibody that binds to T-cells and a tumor antigen, a cellular immune modulator such as a CAR-T cell, a vaccine, an oncolytic virus, and any combination thereof. In some embodiments, the inhibitor to the inhibitory checkpoint molecule is a small molecule drug, or an antibody or a fragment thereof that specifically binds to the inhibitory checkpoint molecule and inhibits its activity, wherein the inhibitory checkpoint molecule is selected from the group consisting of PD-1, PD-L1, PD-L2, CTLA-4, BTLA, A2aR, B7-H2, B7-H3, B7-H4, B7-H6, CD47, CD48, CD160, CD244 (2B4), CHK1, CHK2, CGEN-15049, ILT-2, ILT-4, LAG-3, VISTA, gp49B, PIR-B, TIGIT, TIM1, TIM2, TIM3, TIM4, and KIR, and ligands thereof. In other embodiments, the activator of the stimulatory checkpoint molecule is a small molecule drug, polypeptide-based activator, or polynucleotide-based activator that specifically binds to the stimulatory checkpoint molecule and increases its activity, wherein the stimulatory checkpoint molecule is selected from the group consisting of B7-1 (CD80), B7-2 (CD86), 4-1BB (CD137), OX-40 (CD134), HVEM, inducible costimulator (ICOS), glucocorticoid-induced tumor necrosis factor receptor (GITR), CD27, CD28, CD40, and ligands thereof. In certain embodiments, the chemokine inhibitor is a small molecule drug, or antibody or fragment thereof that specifically binds to the chemokine (or its receptor) and inhibits chemokine activity. In some embodiments, the chemokine is selected from the group consisting of CCL2, CCL3, CCL4, CCL5, CCL7, CCL8, CCL11, CCL12, CCL13, CCL14, CCL15, CCL16, CCL17, CCL18, CCL19, CCL20, CCL21, CCL22, CCL23, CCL24, CCL5, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL5, and CXCL16. In some embodiments, the chemokine inhibitor binds to a chemokine receptor selected from the group consisting of CCR1, CCR2, CCR3, CCR, 4, CCR5, CCR6, CCR7, CCR8, CCR9, CCR10, CXCR1, CXCR2, CXCR3, CXCR4, CXCR5, CXCR6, and CXCR7. The inhibitor of MIF can be a small molecule drug, or antibody or fragment thereof that specifically binds to MIF and inhibits MIF activity. Other inhibitors of macrophage migration can also be used. In some embodiments, the immune modulator is an inhibitor of indoleamine 2, 3-dioxygenase (IDO).

The method can further include: (a) detecting an expression level of one or more biomarkers in a tumor sample from the subject, wherein the one or more biomarkers, e.g., 1, 2, 3, 4, 5 or more biomarkers are selected from the group consisting of an immune cell marker(s), tumor cell marker(s), circulating marker(s), and any combination thereof; (b) comparing the expression level of the one or more biomarkers, e.g., 1, 2, 3, 4, 5 or more biomarkers to the expression level of the one or more biomarkers, e.g., 1, 2, 3, 4, 5 or more biomarkers in a normal tissue sample; and (c) treating the tumor with ionizing radiation and an immune modulator if the expression level of the one or more biomarkers, e.g., 1, 2, 3, 4, 5 or more biomarkers is modified compared to the expression level in the normal tissue sample. In some instances, the expression level of the one or more biomarkers, e.g., 1, 2, 3, 4, 5 or more biomarkers is modified if the expression level of at least one of the biomarkers is increased, or the expression level of at least one of the biomarkers is decreased, or the expression level of at least one of the biomarkers is increased and the expression level of at least one of the biomarkers is decreased compared to the expression level in a normal tissue sample. The expression level of the one or more biomarkers, e.g., 1, 2, 3, 4, 5 or more biomarkers can be ranked or weighted.

Optionally, the method further comprises performing functional imaging of the tumor prior to administering the ionizing radiation and the immune modulator.

In some embodiments, the immune cell biomarker(s) or the tumor cell biomarker(s) or the circulating biomarker(s) is a polynucleotide or a protein. The step of detecting can be performed by using an assay selected from the group consisting of immunohistochemistry, ELISA, Western analysis, HPLC, proteomics, PCR, RT-PCR, Northern analysis, and a microarray.

The tumor sample can be a biopsy comprising tumor cells. The normal tissue sample can comprise non-tumor cells from the same tissue type as the tumor.

The ionizing radiation is administered at a higher dose compared to a standard treatment protocol if the expression level of the one or more biomarkers, e.g., 1, 2, 3, 4, 5 or more biomarkers in the tumor sample is modified compared to the expression level in the normal tissue sample. In certain instances, the ionizing radiation is administered as a hypofractionated radiation treatment if the expression level of the one or more biomarkers, e.g., 1, 2, 3, 4, 5 or more biomarkers in the tumor sample is modified compared to the expression level in the normal tissue sample. In other instances, the ionizing radiation is administered as a hyperfractionated radiation treatment if the expression level of the one or more biomarkers, e.g., 1, 2, 3, 4, 5 or more biomarkers in the tumor sample is modified compared to the expression level in the normal tissue sample.

The ionizing radiation and the immune modulator can be administered concomitantly. Alternatively, the ionizing radiation and the immune modulator can be administered sequentially.

In another aspect, provided herein is a method of treating a tumor in a subject with cancer comprising: (a) determining an expression level of one or more biomarkers, e.g., 1, 2, 3, 4, 5 or more biomarkers in a tumor sample from the subject, wherein the one or more biomarkers, e.g., 1, 2, 3, 4, 5 or more biomarkers are selected from the group consisting of an immune cell marker(s), tumor cell marker(s), circulating marker(s), and any combination thereof; (b) comparing the expression level of the one or more biomarkers, e.g., 1, 2, 3, 4, 5 or more biomarkers to an expression level of the one or more biomarkers, e.g., 1, 2, 3, 4, 5 or more biomarkers in a normal tissue sample; and (c) administering to the tumor in the subject a treatment comprising ionizing radiation and an immune modulator if the expression level of the one or more biomarkers, e.g., 1, 2, 3, 4, 5 or more biomarkers in the tumor sample is modified compared to the expression level in the normal tissue sample.

In some embodiments, the expression level of the one or more biomarkers, e.g., 1, 2, 3, 4, 5 or more biomarkers is modified if the expression level of at least one of the biomarkers is increased, or the expression level of at least one of the biomarkers is decreased, or the expression level of at least one of the biomarkers is increased and the expression level of at least one of the biomarkers is decreased compared to the expression level in a normal tissue sample. The expression level of the one or more biomarkers, e.g., 1, 2, 3, 4, 5 or more biomarkers can be ranked or weighted.

In some instances, the step of administering ionizing radiation comprises contacting the tumor with a radiosensitizer. The ionizing radiation can be administered at a higher dose compared to a standard treatment protocol if the expression level of the one or more biomarkers, e.g., 1, 2, 3, 4, 5 or more biomarkers in the tumor sample is modified compared to the expression level in the normal tissue sample. The ionizing radiation can be administered as a hypofractionated radiation treatment if the expression level of the one or more biomarkers, e.g., 1, 2, 3, 4, 5 or more biomarkers in the tumor sample is modified compared to the expression level in the normal tissue sample. In other cases, the ionizing radiation is administered as a hyperfractionated radiation treatment if the expression level of the one or more biomarkers, e.g., 1, 2, 3, 4, 5 or more biomarkers in the tumor sample is modified compared to the expression level in the normal tissue sample.

The immune modulator can be selected from the group consisting of an inhibitor to an inhibitory checkpoint molecule, an activator of a stimulatory checkpoint molecule, a chemokine inhibitor, an inhibitor of macrophage migration inhibitory factor (MIF), a growth factor, a cytokine, an interleukin, an interferon, an antibody that binds to an immune system cell, a cellular immune modulator, a vaccine, an oncolytic virus, and any combination thereof. The ionizing radiation and the immune modulator are administered concomitantly. In certain instances, the ionizing radiation and the immune modulator are administered sequentially.

The method described herein can also include performing functional imaging of the tumor prior to administering the ionizing radiation and the immune modulator.

In yet another aspect, provided herein is a method of identifying a subject with cancer as a candidate for treatment comprising ionizing radiation and an immune modulator. The method comprises (a) determining an expression level of one or more biomarkers, e.g., 1, 2, 3, 4, 5 or more biomarkers in a tumor sample from the subject, wherein the one or more biomarkers are selected from the group consisting of an immune cell marker(s), tumor cell marker(s), circulating marker(s), imaging marker(s), and any combination thereof (b) comparing the expression level of the one or more biomarkers, e.g., 1, 2, 3, 4, 5 or more biomarkers to an expression level of the one or more biomarkers, e.g., 1, 2, 3, 4, 5 or more biomarkers in a normal tissue sample; and (c) classifying the subject as a candidate for treatment comprising ionizing radiation and the immune modulator if the expression level of the one or more biomarkers, e.g., 1, 2, 3, 4, 5 or more biomarkers in the tumor sample is modified compared to the expression level in the normal tissue sample. In some instances, the expression level of the one or more biomarkers, e.g., 1, 2, 3, 4, 5 or more biomarkers is modified if the expression level of at least one of the biomarkers is increased, or the expression level of at least one of the biomarkers is decreased, or the expression level of at least one of the biomarkers is increased and the expression level of at least one of the biomarkers is decreased compared to the expression level in a normal tissue sample. In certain cases, the expression level of the one or more biomarkers, e.g., 1, 2, 3, 4, 5 or more biomarkers is ranked or weighted. In some cases, the method further comprises performing functional imaging of the tumor.

In some embodiments, the immune modulator is selected from the group consisting of an inhibitor to an inhibitory checkpoint molecule, an activator of a stimulatory checkpoint molecule, a chemokine inhibitor, an inhibitor of macrophage migration inhibitory factor (MIF), a growth factor, a cytokine, an interleukin, an interferon, an antibody that binds to an immune system cell, a cellular immune modulator, a vaccine, an oncolytic virus, and any combination thereof. The ionizing radiation can be administered at a higher dose compared to a standard treatment protocol if the expression level of the one or more biomarkers, e.g., 1, 2, 3, 4, 5 or more biomarkers in the tumor sample is modified compared to the expression level in the normal tissue sample. In some instances, the ionizing radiation is administered as a hypofractionated radiation treatment if the expression level of the one or more biomarkers, e.g., 1, 2, 3, 4, 5 or more biomarkers in the tumor sample is modified compared to the expression level in the normal tissue sample. In other instances, the ionizing radiation is administered as a hyperfractionated radiation treatment if the expression level of the one or more biomarkers, e.g., 1, 2, 3, 4, 5 or more biomarkers in the tumor sample is modified compared to the expression level in the normal tissue sample. The ionizing radiation and the immune modulator are administered concomitantly. The ionizing radiation and the immune modulator are administered sequentially.

In another aspect, provided herein is a method of selecting a treatment for a subject with cancer comprising (a) determining an expression level of one or more biomarkers, e.g., 1, 2, 3, 4, 5 or more biomarkers in a tumor sample from the subject, wherein the one or more biomarkers are selected from the group consisting of an immune cell marker(s), tumor cell marker(s), circulating marker(s), and any combination thereof; (b) comparing the expression level of the one or more biomarkers, e.g., 1, 2, 3, 4, 5 or more biomarkers to an expression level of the one or more biomarkers, e.g., 1, 2, 3, 4, 5 or more biomarkers in a normal tissue sample; and (c) selecting a treatment comprising ionizing radiation and an immune modulator if the expression level of the one or more biomarkers, e.g., 1, 2, 3, 4, 5 or more biomarkers in the tumor sample is modified compared to the expression level in the normal tissue sample. In some embodiments, comprising performing functional imaging of the tumor; and selecting the treatment comprising the ionizing radiation and the immune modulator based on the functional imaging of the tumor. In some cases, the ionizing radiation comprises contacting the tumor with a radiosensitizer.

In some embodiments, the expression level of the one or more biomarkers, e.g., 1, 2, 3, 4, 5 or more biomarkers is modified if the expression level of at least one of the biomarkers is increased, or the expression level of at least one of the biomarkers is decreased, or the expression level of at least one of the biomarkers is increased and the expression level of at least one of the biomarkers is decreased compared to the expression level in a normal tissue sample. The expression level of the one or more biomarkers, e.g., 1, 2, 3, 4, 5 or more biomarkers can be ranked or weighted.

In some embodiments, the immune modulator is selected from the group consisting of an inhibitor to an inhibitory checkpoint molecule, an activator of a stimulatory checkpoint molecule, a chemokine inhibitor, an inhibitor of macrophage migration inhibitory factor (MIF), a growth factor, a cytokine, an interleukin, an interferon, an antibody that binds to an immune system cell, a cellular immune modulator, a vaccine, an oncolytic virus, and any combination thereof. The ionizing radiation can be administered at a higher dose compared to a standard treatment protocol if the expression level of the one or more biomarkers in the tumor sample is modified compared to the expression level in the normal tissue sample. In some cases, the ionizing radiation is administered as a hypofractionated radiation treatment if the expression level of the two or more biomarkers in the tumor sample is modified compared to the expression level in the normal tissue sample. In other cases, the ionizing radiation is administered as a hyperfractionated radiation treatment if the expression level of the one or more biomarkers in the tumor sample is modified compared to the expression level in the normal tissue sample.

In another aspect, a kit is provided. The kit comprises reagents capable of detecting expression of the biomarkers described herein. In some embodiments, the kit comprises reagents capable of detecting nucleic acid (e.g., RNA) expression of the biomarkers. For example, the kit can comprise oligonucleotide primers that are capable amplifying a nucleic acid expressed by the biomarker genes described herein. In some embodiments, the kit further comprises an oligonucleotide probe that hybridizes to a biomarker nucleic acid or an amplified biomarker nucleic acid, or a complement thereof. Methods of amplifying and detecting nucleic acids are well known in the art, and can comprise PCR, RT-PCR real-time PCR, and quantitative real-time PCR, Northern analysis, sequencing of expressed nucleic acids, and hybridization of expressed and/or amplified nucleic acids to microarrays. In some embodiments, the kit comprises reagents that are capable of detecting proteins expression by the biomarkers described herein. In some embodiments, the reagents are antibodies that specifically bind to biomarker proteins. Methods of detecting protein expression are well known in the art, and include immunoassays, ELISA, Western analysis, and proteomic techniques.

In some embodiments of any of the above aspects and embodiments, the differences in the expression levels of each of the biomarkers in the tumor sample are increased or decreased by at least 10%, 20%, 30%, 40%, 50%, 60%, 70%, 80%, 90% or more compared to the expression level in normal tissue. In some embodiments, the expression levels of each of the biomarkers in the tumor sample are increased or decreased by at least 1-fold, 2-fold, 3-fold, 4-fold, 5-fold, 6-fold, 7-fold, 8-fold, 9-fold, 10 fold or more relative to the expression level in normal tissue.

In some embodiments, the average and/or ranked expression level of all the biomarkers in the tumor sample is increased or decreased relative to the expression level in normal tissue. Thus, in some embodiments, the average and/or ranked expression level of all the biomarkers in the tumor sample is increased or decreased by at least 10%, 20%, 30%, 40%, 50%, 60%, 70%, 80%, 90% or more compared to the expression level in normal tissue. In some embodiments, the expression levels in normal tissue are normalized to a control or baseline level. It will be understood that the expression level can also be compared to the expression level in the tumor sample before, after or during a treatment, course of treatment, or treatment plan. Thus, in some embodiments, the expression levels of each of the biomarkers in the tumor sample are increased or decreased by at least 10%, 20%, 30%, 40%, 50%, 60%, 70%, 80%, 90% or more compared to the expression level in the tumor sample before, during or after treatment.

Further, with regard to any of the above aspects and embodiments, the one or more biomarkers can comprise or consist of any combination of the biomarkers, for example, any of the biomarkers described herein, any combination of two or more biomarkers, any combination of three or more biomarkers, any combination of four or more biomarkers, any combination of five or more biomarkers, any combination of six or more biomarkers, and any combination of seven or more biomarkers.

In another aspect, the expression level of at least one, two, three, four or more of the biomarkers described herein is determined. The combination of expression levels of two or more biomarkers, e.g., 2, 3, 4, 5, 6 or more biomarkers can indicate that the subject with cancer is more sensitive to radiation compared to a control subject. This subject may be administered a reduced or decreased dose of radiation compared to a standard dose. In other instances, if the combination of expression levels of two or more biomarkers, e.g., 2, 3, 4, 5, 6 or more biomarkers can indicate that the subject with cancer is less sensitive to radiation compared to a control subject. A subject who is less sensitive to radiation may be administered an increased dose, a hypofractionated dose or a hyperfractionated dose of radiation. Optionally, radiation therapy may be administered in combination with an immune modulator, such as but not limited to, an anti-TIM4 antibody, an anti-MFG-E8 antibody, an anti-M199 antibody, and any combination thereof.

In some embodiments, the biomarker is CD44, MFG-E8, CD68, TGFβ, or any combination thereof. In certain embodiments, if a first biomarker has a high level of expression and a second biomarker has a low level of expression in a sample obtained from a subject with cancer relative to a control sample, then it is predicted that radiation treatment monotherapy may result in local tumor control failure. As such, this biomarker profile can indicate that the subject should be administered radiation treatment in combination with an immune modulator. Alternatively, this biomarker profile can indicate that the dose of radiation be increased (i.e., increased over a standard protocol dose). For instance, if the level of CD44 is high and the level of MFG-E8 is low in a subject's tumor sample compared to a control sample, then it is predicted that radiation treatment alone will not lead to a clinical response. In other words, a tumor sample having a high level of CD44 and a low level of MFG-E8 is likely to be insensitive or have a low sensitivity to ionizing radiation therapy. In some cases, the biomarker profile described herein indicates that the subject should receive an increased dose of radiation and/or combination therapy comprising ionizing radiation and an immune modulator, such as an anti-TIM4 antibody, anti-MFG-E8 antibody, anti-M199 antibody, and any combination thereof

In other embodiments, if the level of CD44 is low compared to a normal sample and/or the level of MFG-E8 is high compared to a normal sample, the subject is likely to have a clinical response to ionizing radiation monotherapy. In some cases, it is predicted that a subject with low level of CD44 and/or a high level of MFG-E8 is likely to be sensitive to ionizing radiation therapy.

In some embodiments, if a subject's tumor has a high level of CD68 compared to a control sample, the subject is predicted to have decreased survival after radiation monotherapy. As such, this subject can be administered a combination therapy comprising ionizing radiation and an immune modulator. In other instances, if a subject's tumor has a low level of CD68 compared to a control sample, the subject is likely to have a clinical response to radiation monotherapy. It is predicted that this subject is sensitive to radiation. In certain cases, it may be indicated that the subject be administered a low dose or reduced dose of radiation compared to a standard protocol dose.

A. Biomarkers for Therapy Selection

The biomarkers described herein can be used to stratify patients to receive individualized, tailored radiotherapy in combination with an immune modulator agent. The biomarkers can also be used to monitor the efficacy of immune modulator therapy on patients with cancer. The biomarkers include, but are not limited to, one or more immune cell biomarkers, one or more tumor cell biomarkers, one or more circulating biomarkers, one or more imaging biomarkers, and any combination thereof. For instances, an immune cell biomarker can provide information about the location and/or activity of a specific cell population, such as a T cell population. An immune cell biomarker or tumor cell biomarker can be a genetic biomarker, polynucleotide biomarker, or a protein biomarker. In some embodiments, an immune cell biomarker is a specific polynucleotide (e.g., RNA and microRNA) or protein that is expressed at a higher level by a particular immune cell compared to a non-immune cell or a different type of immune cell. Similarly, a tumor cell biomarker can a specific polynucleotide (e.g., RNA and microRNA) or protein that is expressed at a higher level by a tumor cell compared to a non-tumor cell. For example, the tumor cell biomarker can be a protein or a polynucleotide encoding said protein that is associated with proliferation and/or metastasis of a tumor cell. In some cases, the protein can be involved in angiogenesis or other processes that are activated by a tumor cell. The tumor biomarker can be an oncogene or a tumor suppressor. In some instances, a tumor cell biomarker is a gene variation, gene mutation, copy number variant (CNV), single nucleotide polymorphism (SNP), and the like that is present in a tumor cell, but not in a non-tumor cell. In some embodiments, a circulating biomarker is an exosome (i.e., a cell-derived vesicle that can be found in a body fluid). Examples of useful biomarkers includes those described in U.S. Patent Appl. Publ. No. 20160024594, the disclosure of which is hereby incorporated by reference for all purposes.

The biomarker set can include, but is not limited to, CD44, milk fat globule-EGF factor 8 (MFG-E8), CD68 and TGFβ. CD44 is a cell-surface glycoprotein that plays a role in cell proliferation, cell-cell interactions, cell adhesion, and cell migration of various cell types including lymphocytes and cancer cells. The human CD44 polypeptide sequence is set forth in, e.g., GenBank Accession No. NP_000601. The human CD44 mRNA (coding) sequence is set forth in, e.g., GenBank Accession No. NM_000610. Milk fat globule-EGF factor 8 protein (MFG-E8) is a macrophage-produced protein that promotes engulfment and clearance of apoptotic cells in tumors. The human MFG-E8 polypeptide sequence is set forth in, e.g., GenBank Accession No. NP_005919. The human MFG-E8 mRNA (coding) sequence is set forth in, e.g., GenBank Accession No. NM_005928. CD68 is a 110-kD transmembrane glycoprotein that is highly expressed by human monocytes and tissue macrophages. The protein primarily localizes to lysosomes and endosomes with a smaller fraction circulating to the cell surface. It is a type I integral membrane protein with a heavily glycosylated extracellular domain and binds to tissue- and organ-specific lectins or selectins. CD68 is also a member of the scavenger receptor family. The human CD68 polypeptide sequence is set forth in, e.g., GenBank Accession No. NP_001242. The human CD68 mRNA (coding) sequence is set forth in, e.g., GenBank Accession No. NM_001251. TGFβ is a cytokine that is involved in cell growth, cell proliferation, cell differentiation, apoptosis, homeostasis and many other cellular processes. The human TGFβ polypeptide sequence is set forth in, e.g., GenBank Accession No. NP_000651. The human TGFβ mRNA (coding) sequence is set forth in, e.g., GenBank Accession No. NM_000660.

It will be understood that the expression levels of each of the biomarkers described herein in the patient sample can increase or decrease relative to the expression level of the tumor biomarker in a normal or control tissue sample. For example, the expression level of one tumor biomarker can increase in the tumor sample compared to the expression level in a normal tissue, whereas the expression level of a second biomarker can decrease in the tumor sample compared to the expression level in a normal tissue. The expression level can also be based on the average, combination or sum of the all the tumor biomarker expression levels in the patient sample. For example, the expression level of each biomarker in the patient sample can be ranked or weighted to produce a ranked value that is higher or lower than the normal tissue value (which can be a normalized value, for example, set to 1).

In some embodiments, biomarker expression is determined in a biological sample from the subject having a tumor. In some embodiments, the biological sample is a tumor sample. The tumor sample can be a biopsy comprising tumor cells from the tumor. In some embodiments, the biological sample comprises a bodily fluid, such as but not limited to blood, serum, plasma, or urine, and/or cells or tissues from the subject. In some embodiments, the biological sample is a formalin-fixed and paraffin embedded tissue or tumor sample. In some embodiments, the biological sample is a frozen tissue or tumor sample. Thus, in some embodiments, one or more steps of the methods described herein are carried out in vitro. For example, in some embodiments, biomarker expression is determined in vitro.

In some embodiments, the normal tissue sample comprises non-tumor cells from the same tissue type as the tumor. In some embodiments, the normal tissue sample is obtained from the same subject diagnosed with the tumor. A normal tissue sample can also be a control sample of the same tissue-type from a different subject. The expression level of the normal tissue sample can also be an average or mean value obtained from a population of normal tissue samples.

The level of expression of the biomarkers described herein can be determined using any method known in the art. For example, the level of expression can be determined by detecting the expression of a nucleic acid (e.g., RNA, mRNA or microRNA) or the protein encoded by the nucleic acid.

Exemplary methods for detecting expression levels of nucleic acids include, without limitation, Northern analysis, polymerase chain reaction (PCR), reverse transcription PCR (RT-PCR), real-time PCR, quantitative real-time PCR, and DNA microarrays.

Exemplary methods for detecting expression levels of proteins (e.g., polypeptides) include, without limitation, immunohistochemistry, ELISA, Western analysis, HPLC, and proteomics assays. In some embodiments, the protein expression level is determined by immunohistochemistry using the Allred method to assign a score (see, e.g., Allred, D. C., Connection 9:4-5, 2005, which is incorporated by reference herein). For example, formalin-fixed, paraffin embedded tissues are contacted with an antibody that specifically binds a biomarker described herein. The bound antibody is detected with a detectable label or secondary antibody coupled with a detectable label, such as a colorimetric label (e.g., an enzymatic substrate produce by HRP or AP). The antibody positive signal is scored by estimating the proportion of positive tumor cells and their average staining intensity. Both the proportion and intensity scores are combined into a total score that weighs both factors.

In some embodiments, the protein expression level is determined by digital pathology. Digital pathology methods include scanning images of tissues on a solid support, such as a glass slide. The glass slides are scanned into whole slide images using a scanning device. The scanned images are typically stored in an information management system for archival and retrieval. Image analysis tools can be used to obtain objective quantitative measurements from the digital slides. For example, the area and intensity of immunohistochemical staining can be analyzed using the appropriate image analysis tools. Digital pathology systems can include scanners, analytics (visualization software, information management systems and image analysis platforms), storage and communication (sharing services, software). Digital pathology systems are available from numerous commercial suppliers, for example. Aperio Technologies, Inc. (a subsidiary of Leica Microsystems GmbH), and Ventana Medical Systems, Inc. (now part of Roche). Expression levels can be quantified by commercial service providers, including Flagship Biosciences (CO), Pathology, Inc. (CA), Quest Diagnostics (NJ), and Premier Laboratory LLC (CO).

In some embodiments, imaging of the tumor, such as functional imaging is also used to identify or select a cancer patient who should receive the combination therapy described herein. Non-limiting examples of functional imaging include single-photon emission computed tomography, optical imaging, ultrasonography, positron emission tomography (PET), computed tomography (CT), perfusion computed tomography, magnetic resonance imaging (MRI), functional magnetic resonance imaging, magnetic resonance sectroscopic imaging, dynamic contrast-enhanced imaging, diffusion-weighted imaging, blood-oxygenation level dependent imaging, magnetic resonance spectroscopy, magnetic resonance lymphography, and any combination thereof. Any type of functional imaging such as multimodality imaging can be performed to characterize the tumor, to determine the delineation of the tumor, the extent of the tumor, the tumor volume, and/or to assess the tumor microenvironment (e.g., the environment surrounding the tumor). Functional imaging can aid in selecting the best treatment option and/or in monitoring response to the treatment.

B. Methods for Selecting a Course of Treatment

The expression levels of the biomarkers can be used to determine or select a course of treatment in a subject diagnosed with a tumor. For example, in some embodiments, the treatment comprises adminstering ionizing radiation to the tumor in the subject. The ionizing radiation can also be administered to the entire subject or a portion thereof, especially if the tumor is dispersed or mobile. In some embodiments, the treatment further comprises contacting the tumor with a radiosensitizer. In some embodiments, the treatment further comprises administering a compound or biologic drug, such as an antibody, that inhibits an immune checkpoint pathway to the subject. Thus, in some embodiments, the treatment comprises administering a standard radiation treatment protocol in combination with an immune modulator.

The course of treatment can be selected based on the expression levels of the biomarkers. For example, the expression levels can be used to determine if radiation therapy is appropriate for the subject (i.e., for making a go/no go decision on radiotherapy). Further, if the expression levels of the biomarkers are increased relative to a normal or control value, then the effective radiation dose to the tumor can be increased, and/or the fractionation schedule modified accordingly. The radiation dose to the blood vessels feeding the tumor can also be increased. In some cases, a hypofractionated radiation treatment is administered. Alternatively, a hyperfractionated radiation treatment is administered. Optionally, radiation treatment is provided in combination with immune modulator treatment.

In some embodiments, if the expression levels of the biomarkers are increased relative to a normal or control value, then the treatment can comprise administering ionizing radiation to the tumor. In some embodiments, if the expression levels of the biomarkers are decreased relative to a normal or control value, then the treatment can comprise decreasing the amount of ionizing radiation administered to the tumor. Optionally, radiation treatment is provided in combination with immune modulator treatment.

The treatment can also comprise modifying an existing course of treatment. For example, in some embodiments, the existing course of treatment is modified to increase the effective dose of the ionizing radiation administered to the tumor. In some embodiments, the effective dose of ionizing radiation is increased by increasing the amount of ionizing radiation administered to the tumor and/or contacting the tumor with a radiosensitizer. In some embodiments, the existing course of treatment is modified to decrease the effective dose of the ionizing radiation administered to the tumor. In some embodiments, the treatment comprises modifying a standard radiation treatment protocol in combination with administering an immune modulator.

In some embodiments, the effective dose of ionizing radiation administered to the tumor is increased if the level of one or more biomarkers described herein is elevated in the tumor environment. For example, the effective dose of ionizing radiation is increased as compared to the standard of care for a subject that does not have elevated levels of the biomarker(s) in the tumor environment. This applies to subjects who are currently not undergoing radiation therapy as well as modifying an existing course of treatment for subjects undergoing radiation therapy. Thus, the effective dose of ionizing radiation can be increased from the current effective dose if the subject is already undergoing radiation therapy for a tumor. The radiation therapy can be modified to reduce the constraints on neighboring healthy tissue. For example, if the biomarker level in the tumor environment indicates more aggressive radiation therapy is required, the treatment plan can be modified so that the constraints on the border between healthy tissue and tumor tissue are decreased. This would result in a trade-off between damaging some healthy tissue in order to kill more of the tumor tissue.

In some embodiments, the treatment comprises a combination of radiation therapy and an immune modulator agent (including a radiosensitizer). In some embodiments, the effective dose of ionizing radiation administered to the tumor is not changed (e.g., relative to the standard of care or relative to an existing course of treatment) when an immune modulator agent is administered to the subject. For example, in some embodiments, the subject is administered an effective dose of ionizing radiation that is the same or similar to that administered to a subject that does not have elevated levels of one or more biomarkers described herein in the tumor environment, and the subject is further administered an immune modulator agent. In some embodiments, the effective dose of ionizing radiation administered to the tumor is based on the standard of care for a subject that does not have elevated levels of the biomarker(s) in the tumor environment, and the subject is further administered an immune modulator agent. In some embodiments involving an existing course of treatment, the effective dose of ionizing radiation is maintained at the current effective dose, and an anti-cancer agent is administered to the subject in combination with the ionizing radiation if the level of one or more biomarkers described herein is elevated in the tumor environment.

In some embodiments, the treatment plan is developed and/or modified based on the expression levels of the biomarkers described herein.

The course of treatment can also be selected by using an algorithm that determines the expression level of the biomarkers in the tumor sample relative to the level in the normal sample. The algorithm can be a linear regression algorithm that includes the biomarker expression levels and coeffcients (i.e., weights) for combining the expression levels. In some embodiments, the algorithm comprises a least squares fit to calculate the coefficients. If the algorithm determines that the expression level of the biomarkers in the tumor sample is increased or decreased relative to the normal sample, then the appropriate course of treatment can be assigned. In some embodiments, the algorithm is a nonparametric regression tree. In some embodiments, standard statistical methods were used to analyze the data to determine which biomarkers were most predictive of clinical survival or local tumor control failure.

In some embodiments, the method described herein is a computer implemented method. In some embodiments, the computer implemented method comprises a linear regression model that assigns a ranked or weighted value to the expression levels of the biomarkers desribed herein. In some embodiments, the disclosure provides a computer-readable medium, the medium providing instructions to cause a computer to perform a method described herein. For example, the medium can provide instructions to cause a computer to assign a ranked or weighted value to the expression levels of the biomarkers desribed herein.

C. Radiation Therapy

The expression levels of the tumor biomarkers described herein can be used to optimize treatment of patients with radiotherapy. For example, the therapeutic dose of the radiation adminstered to the tumor or subject can be adjusted based on the expression levels of the biomarkers. As is well known in the art, the effective dose of ionizing radiation varies with the type of tumor and stage of cancer that needs to be treated. The effective dose can also vary based on other treatment modalities being administered to the patient, for example chemotherapeutic treatments and surgical treatments, and whether the radiation is administered pre- or post-surgery. In general, a curative therapeutic dose for a solid epithelial tumor ranges from about 60 to 80 gray (Gy), whereas a curative dose for a lymphoma is about 20 to 40 Gy. In general, preventative doses can be 45-60 Gy.

As is well known in the art, the therapeutic dose can be delivered in fractions. Fractionation refers to spreading out the total dose of radiation over time, for example, over days, weeks or months. The dose delivered in each fraction can be about 1.5-2 Gy per day. The treatment plan can include a fraction treatment one or more times per day, every other day, weekly, etc. depending on the treatment needs of each patient. For example, a hypofractionation schedule comprises dividing the total dose into several relativley large doses, and administering the doses at least one day apart. Exemplary hypofraction doses are 3 Gy to 20 Gy per fraction. An exemplary fractionation schedule that can be used to treat lung cancer is Continuous Hyperfractionated Accelerated Radiation therapy (CHART), which consists of three small fractions per day.

In some embodiments, the ionizing radiation includes contacting the tumor in the subject with a radiosensitizer. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. SSB repair mechanisms include BER, NER, or MMR pathways whilst DSB repair mechanisms consist of HR and NHEJ pathways. Radiation causes DNA breaks that if not repaired are lethal. Single strand breaks are repaired through a combination of BER, NER and MMR mechanisms using the intact DNA strand as a template. The predominant pathway of SSB repair is the BER utilizing a family of related enzymes termed poly-(ADP-ribose) polymerases (PARP). Thus, the radiosensitizer can include DNA damage response inhibitiors such as Poly (ADP) ribose polymerase (PARP) inhibitors.

The biomarkers described herein are useful in developing and modifying treatment plans for patients diagnosed with a tumor or cancer. The treatment plan can include visualizing or measuring the tumor volume that needs to be irradiated, the optimal or effective dose of radiation administered to the tumor, and the maximum dose to prevent damage to nearby healthy tissue or organs at risk. Algorithms can used in treatment planning, and include dose calculation algorithms based on the particular radiotherapy technique parameters employed, e.g., gantry angle, MLC leaf positions, etc., and search algorithms which use various techniques to adjust system parameters between dose calculations to optimize the effectiveness of the treatment. Exemplary dose calculation algorithms include various Monte Carlo (“MC”) techniques and pencil beam convolution (“PBC”). Exemplary search algorithms include various simulated annealing (“SA”) techniques, algebraic inverse treatment planning (“AITP”), and simultaneous iterative inverse treatment planning (“SIITP”). Such techniques, and others, are well known in the art, and are included within the scope of this disclosure.

Treatment planning algorithms may be implemented as part of an integrated treatment planning software package which provides additional features and capabilities. For example, a dose calculation algorithm and search algorithm may be used to optimize a set of fluence maps at each gantry angle, with a separate leaf sequencer used to calculate the leaf movements needed to deliver them. Alternatively, a dose calculation algorithm and search algorithm may be used to directly optimize leaf movements and other machine parameters. The Eclipse™ Treatment Planning System offered by the assignee of the present invention includes such an integrated software program. Methods for optimizing treatment plans are described in U.S. Pat. No. 7,801,270, which is incorporated by reference herein.

In some embodiments, the biomarkers described herein can be used to monitor the progress of tumor control after radiation therapy. For example, the expression levels of the biomarkers before and after ionizing radiation therapy can be compared. In some embodiments, if the expression levels of biomarkers increase after radiotherapy, this suggests that the tumor is continuing to grow in size. Thus, the radiation treatment can be modified based on monitoring tumor growth using the biomarkers described herein.

The biomarkers described herein can be used with any radiation therapy technique known in the art. Radiation therapy techniques include external-beam radiotherapy (“EBRT”) and Intensity Modulated Radiotherapy (“IMRT”), which can be administered by a radiotherapy system, such as a linear accelerator, equipped with a multileaf collimator (“MLC”). The use of multileaf collimators and IMRT allows the patient to be treated from multiple angles while varying the shape and dose of the radiation beam, thereby avoiding excess irradiation of nearby healthy tissue. Other exemplary radiation therapy techniques include stereotactic body radiotherapy (SBRT), volumetric modulated arc therapy, three-dimensional conformal radiotherapy (“3D conformal” or “3DCRT”), image-guided radiotherapy (IGRT). The radiation therapy techniques can also include Adaptive radiotherapy (ART), a form of IGRT that can revise the treatment during the course of radiotherapy in order to optimize the dose distribution depending on patient anatomy changes, and organ and tumor shape. Another radiation therapy technique is brachytherapy. In brachytherapy, a radioactive source is implanted within the body of the subject, such that the radioactive source is near the tumor. As used herein, the term radiotherapy should be broadly construed and is intended to include various techniques used to irradiate a patient, including use of photons (such as high energy x-rays and gamma rays), particles (such as electron and proton beams), and radiosurgical techniques. Further, any method of providing conformal radiation to a target volume is intended to be within the scope of the present disclosure.

D. Immune Modulators

The radiation therapy can be administered in combination with one or more immune modulators. The combination therapy can provide an increased anti-tumor response (a positive clinical response) compared to administration of either treatment as monotherapy. In some cases, the immune modulator can be selected from the group consisting of an inhibitor to an inhibitory checkpoint molecule, an activator of a stimulatory checkpoint molecule, a chemokine inhibitor, an inhibitor of macrophage migration inhibitory factor (MIF), a growth factor, a cytokine, an interleukin, an interferon, an antibody that binds to an immune system cell, such as a bispecific antibody that binds to T-cells and a tumor antigen, a cellular immune modulator such as a CAR-T cell, a vaccine, an oncolytic virus, and any combination thereof.

Immune modulators can include small molecules and biologic therapies (e.g., antibodies, fragments thereof, and derivatives thereof) that bind molecules expressed on the surface of immune system cells, such as antigen presenting cells and T-cells. Immune modulators also can include small molecules that inhibit or stimulate the immune system. In some instances, the immune modulator stimulates CD27+ immune cells, or inhibits one or more inhibitory checkpoint molecule(s) including PD-1, PD-L1, PD-L2, CTLA-4, BTLA, A2aR, B7-H2, B7-H3, B7-H4, B7-H6, CD47, CD48, CD160, CD244 (2B4), CHK1, CHK2, CGEN-15049, ILT-2, ILT-4, LAG-3, VISTA, gp49B, PIR-B, TIGIT, TIM1, TIM2, TIM3, TIM4, KIR, and ligands thereof, and others. Immune checkpoint pathways and signaling molecules are described in, e.g., Pardoll, Nature Rev Cancer, 2012, 12:252-264; and Mellman et al., Nature, 2011, 480:480-489.

An inhibitor of an inhibitory checkpoint molecule can be an antibody or fragment thereof that specifically binds or recognizes PD-1, PD-L1, PD-L2, CTLA-4, BTLA, A2aR, B7-H2, B7-H3, B7-H4, B7-H6, CD47, CD48, CD160, CD244 (2B4), CHK1, CHK2, CGEN-15049, ILT-2, ILT-4, LAG-3, VISTA, gp49B, PIR-B, TIGIT, TIM1, TIM2, TIM3, TIM4, KIR, and ligands thereof. In some embodiments, the CTLA-4 inhibitor is selected from the group consisting of ipilimumab, tremelimumab, and the like. One non-limiting example of a small molecule immune modulator is an inhibitor of the enzyme indolamine 2,3-dioxygenase (IDO). In some embodiments, the immune modulator is an inhibitor of PD-1, PD-L1, PD-L2, or CTLA-4.

In some embodiments, the PD-1 inhibitor is selected from the group consisting of pembrolizumab, nivolumab, lambrolizumab, pidilizumab, AMP-244, MEDI-4736, MPDL328 OA, MIH1, IBI-308, mDX-400, BGB-108, MEDI-0680, SHR-1210, PF-06801591, PDR-001, GB-226, STI-1110, biosimilars thereof, biobetters thereof, and bioequivalents thereof. In some embodiments, the PD-L1 inhibitor is selected from the group consisting of durvalumab, atezolizumab, avelumab, BMS-936559, ALN-PDL, TSR-042, KD-033, CA-170, STI-1014, KY-1003, biosimilars thereof, biobetters thereof, and bioequivalents thereof.

In some embodiments, the activator of the stimulatory checkpoint molecule is a small molecule, antibody or a fragment thereof, a polypeptide-based activator, a polynucleotide-based activator (i.e., an aptamer), agonist, agonist antibody or fragment thereof, and the like. The stimulatory checkpoint molecule can be B7-1 (CD80), B7-2 (CD86), 4-1BB (CD137), OX40 (CD134), HVEM, inducible costimulator (ICOS), glucocorticoid-induced tumor necrosis factor receptor (GITR), CD27, CD28, CD40, or a ligand thereof.

In some embodiments, a chemokine inhibitor is administered as an immune modulator. The chemokine inhibitor can be a small molecule, or antibody or fragment thereof that specifically binds to the chemokine (or its receptor) and inhibits its activity. In some embodiments, the chemokine is selected from the group consisting of CCL2, CCL3, CCL4, CCL5, CCL7, CCL8, CCL11, CCL12, CCL13, CCL14, CCL15, CCL16, CCL17, CCL18, CCL19, CCL20, CCL21, CCL22, CCL23, CCL24, CCL5, CCL26, CCL27, CCL28, CXCL1, CXCL2, CXCL3, CXCL4, CXCL5, CXCL6, CXCL7, CXCL8, CXCL9 , CXCL10, CXCL11, CXCL12, CXCL13, CXCL14, CXCL5, and CXCL16, or any other chemokine that is associated with cancer such as trafficking leukocytes into the tumor microenvironment (e.g., control leukocyte infiltration to the tumor). In some embodiments, the chemokine inhibitor binds to a chemokine receptor selected from the group consisting of CCR1, CCR2, CCR3, CCR, 4, CCR5, CCR6, CCR7, CCR8, CCR9, CCR10, CXCR1, CXCR2, CXCR3, CXCR4, CXCR5, CXCR6, and CXCR7.

Additional examples of an immune modulator include but are not limited to an anti-TIM4 antibody, an anti-MFG-E8 antibody, an anti-M199 antibody, any combination thereof, and the like. In some embodiments, the immune modulator includes agents (antibodies or small molecules) involved in priming and activation of the immune systems, and includes agents targeting CTLA4, B7 (B7-1or B7-2), PD-L1/PD-L2, or PD-1, or agents targeting the binding interactions between CTLA4 and B7-1/B7-2, or PD-1 and PD-L1/PD-L2. Agents targeting CTLA4, B7 (B7-1or B7-2), PD-L1/PD-L2, and PD-1 include antibodies that specifically bind these molecules, such as monoclonal antibodies. In some embodiments, the agent is an antibody that specifically binds to LAG 3, TIM1, TIM3, MFG-E8, IL-10, or Phosphatidylserine.

The immune modulators described herein can be administered at therapeutically effective doses. Therapeutically effective doses can be determined by one of ordinary skill in the art based on the type of immune modulator administered. Dosage, routes of administration, and administration schedules described in the art can be used. Representative doses are available in the Merck Manual Professional Edition (see the internet at merckmanuals.com/professional).

Further, doses of immune modulators administered to animals can be converted to equivalent doses for humans based on the body surface area (BSA) (represented in mg/m2) normalization method (see, e.g., Reagan-Shaw, S. et al., “Dose translation from animal to human studies revisited,” FASEB J. 22, 659-661 (2007); and “Guidance for Industry—Estimating the Maximum Safe Starting Dose in Initial Clinical Trials for Therapeutics in Adult Healthy Volunteers,” U.S. Department of Health and Human Services, Food and Drug Administration, Center for Drug Evaluation and Research (CDER), July 2005, Pharmacology and Toxicology; which are incorporated by reference herein). For example, the human equivalent dose (HED) based on BSA is can be calculated by the following formula I:

HED=animal dose in mg/kg×(animal weight in kg/human weight in kg)0.33 I.

Alternatively, the HED can be determined by the following formula II:

HED (mg/kg)=animal dose (mg/kg)×(animal Km/human Km) II.

The Km factor is determined based on the following Table (see Guidance for Industry, Id.):

TABLE 1
Conversion of Animal Doses to Human Equivalent Doses Based on Body
Surface Area

| | To Convert | | |
|---|---|---|---|
| | Animal Dose in | To Convert Animal Dose in mg/kg | |
| | mg/kg to Dose in | to HED^{a} in mg/kg, Either: | |
| | mg/m², Multiply | Divide | Multiply |
| Species | by kₘ | Animal Dose By | Animal Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| monkey | | | |
| Baboon | 20 | 1.8 | 0.54 |
| Micro-pig | 27 | 1.4 | 0.73 |
| Mini-pig | 35 | 1.1 | 0.95 |

Assumes 60 kg human.

Thus, a 5 mg/kg dose in mice is equivalent to a 0.4 mg/kg dose in a 60 kg human. A 0.4 mg/ml dose in a 60 kg human is equivalent to a dose of 14.8 mg/m2.

In some embodiments, the immune modulators described herein are administered in therapeutically effective amounts for periods of time effective to treat a cancer or tumor. The effective amount of the immune modulators described herein can be determined by one of ordinary skill in the art and includes dosage amounts for a mammal of from about 0.5 to about 200 mg/kg, about 0.5 to about 150 mg/kg, about 0.5 to 100 mg/kg, about 0.5 to about 75 mg/kg, about 0.5 to about 50 mg/kg, about 0.01 to about 50 mg/kg, about 0.05 to about 25 mg/kg, about 0.1 to about 25 mg/kg, about 0.5 to about 25 mg/kg, about 1 to about 20 mg/kg, about 1 to about 10 mg/kg, about 20 mg/kg of body weight, about 10 mg/kg, about 5 mg/kg, about 2.5 mg/kg, about 1.0 mg/kg, or about 0.5 mg/kg of body weight of the immune modulator, or any range derivable therein. In some embodiments, the dosage amounts of the immune modulators are from about 0.01 mg/kg to about 10 mg/kg of body weight. In some embodiments, the dosage amount of the immune modulator is from about 0.01 mg/kg to about 5 mg/kg, or from about 0.01 mg/kg to about 2.5 mg/kg of body weight. The compositions described herein can be administered in a single dose or in the form of individual divided doses, such as from 1 to 4 times per day, or once every 2 days, 3 days, 4 days, 5 days, 6 days, weekly, or monthly. The compositions described herein can also be administered for various treatment cycles, such as 2, 3, 4, 5, 6, 7, 8, 9, 10 treatment cycles. The treatment cycles can be different lengths of time depending on the cancer to be treated, for example, 1, 2, 3, 4, 5, 6, 7, 8, 9, or 10 week treatment cycles. In addition, the effective amount of an immune modulator described herein can be determined during pre-clinical trials and clinical trials by methods known to physicians and clinicians.

### III. EXAMPLES

The following examples are offered to illustrate, but not to limit, the claimed invention.

### Example 1

### Identifying and Using Biomarkers to Predict Response to Radiotherapy

A radiosensitivity index based on the expression level of one or more molecular biomarkers can be used to predict a cancer patient's sensitivity to radiation. Genomic biomarkers and other indicators of the tumor microenvironment can also be used to predict a patient's response to radiotherapy. Additionally, molecular-target based biomarkers such as CD44 and TGFβ may be predictive of tumor response.

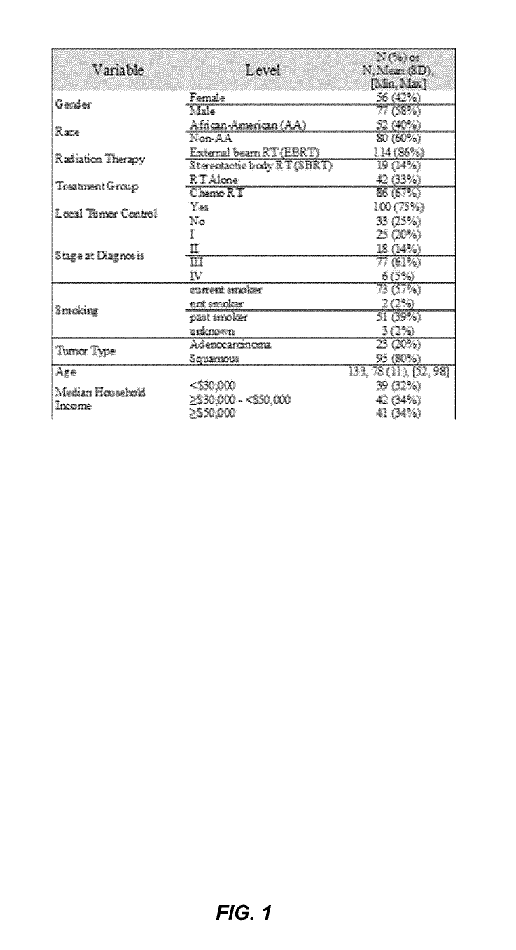

It has been shown that CD44 levels can predict local tumor recurrence after radiotherapy in patients with non-small cell lung cancer (NSCLC). In the study, 133 patients were treated with stereotactic body radiation therapy (SBRT) (12 Gy×4) or conventional fractionated radiation (60 to 70 Gy) (FIG. 1) (see Kumar, S., et al., “Prognostic Biomarkers in Non-Small Cell Lung Cancer Patients Treated With Radiation Therapy: Locally Advanced Non-Small Cell Lung Cancer,” International Journal of Radiation Oncology*Biology*Physics, Volume 90, Issue 5, Supplement, 15 Nov. 2014, Pages S25-S26). Tumor samples were obtained from the patient and stained for specific biomarkers including CD44, MFG-E8, and CD68. Analysis of the biomarker expression revealed that CD44 can be used as a biomarker to predict response to radiotherapy.

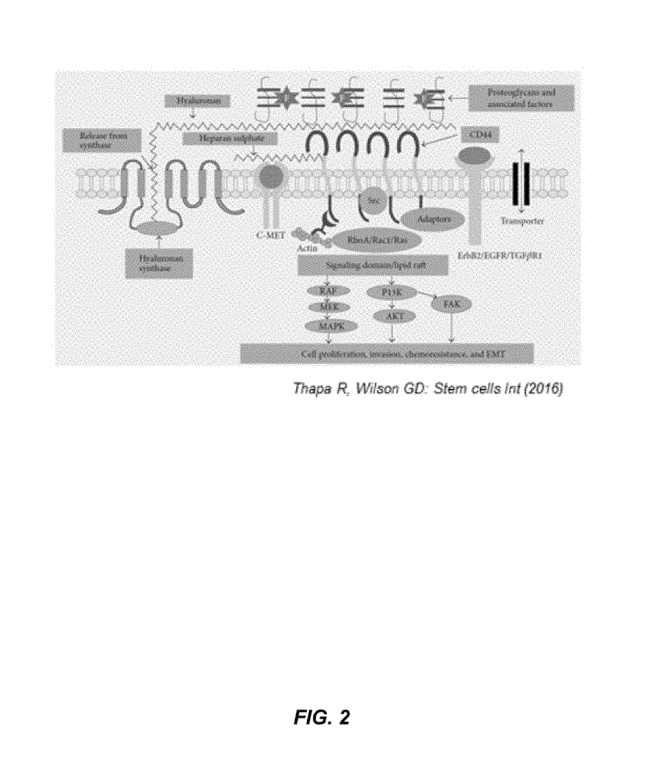

CD44 is a receptor for hyaluronan and is associated with aggressive tumor phenotypes (FIG. 2) (see Thapa R, Wilson G D: Stem cells Int (2016)). It is expressed on cancer initiating cells (CICs) and is involved in TGFβ activation. CD44 has been associated with radioresistance.

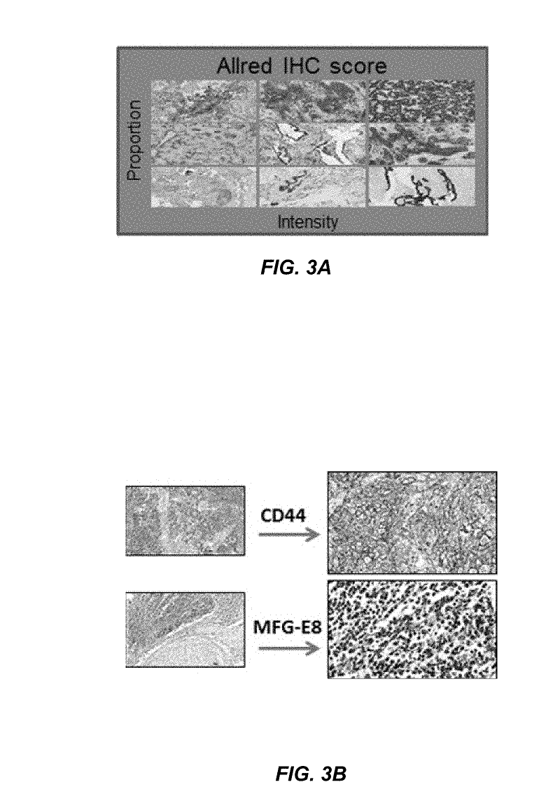

In this study, CD44 protein levesl were quantified according to the Allred scoring system featuring a proportion score and an intensity score to give a total score between 0 and 8 (FIG. 3A). FIG. 3B shows IHC staining of tumor samples with CD44 and MFG-E8. High expression of CD44 and low expression of MFG-E8 were predictive of local tumor control failure. High expression of CD68 was associated with decreased survival benefit to radiotherapy.

Data suggests that the tumor microenvironment may play a role in tumor response to radiotherapy. As such, biomarkers of this microenvironment may be predictive of clinical response.

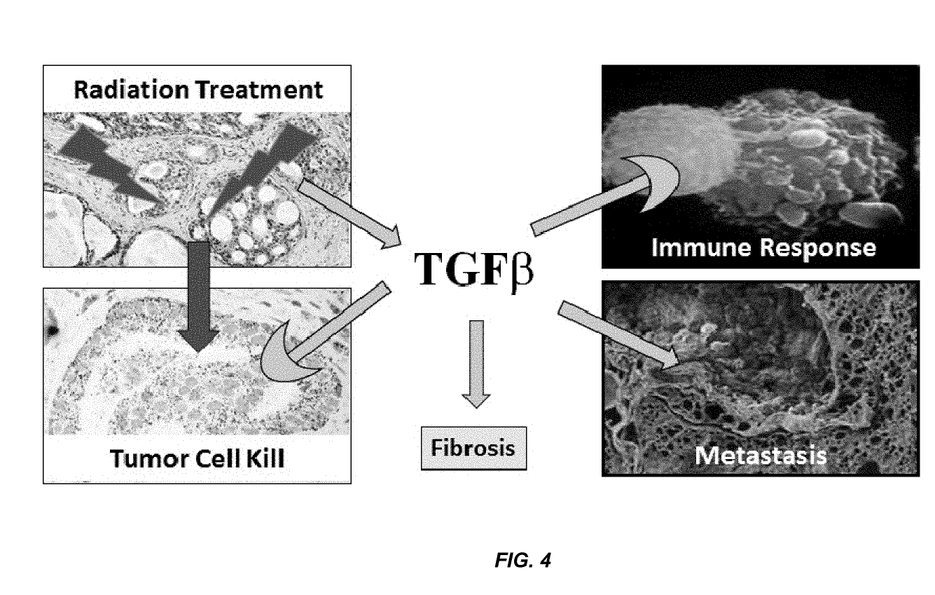

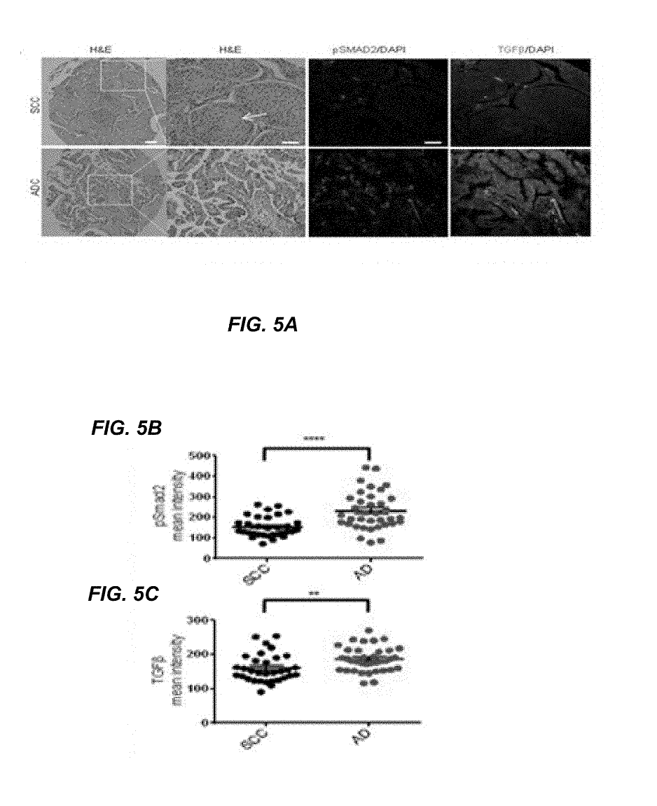

TGFβ is a pleitropic cytokine that is important in normal tissue homeostasis, regulates inflammation and immune responses, and controls proliferation and differentiation. As shown in FIG. 4, there is substantial evidence that TGFP plays a key role in the response to ionizing radiation. TGFP is activated in irradiated tissues and plays a role in development of radiation induced fibrosis. It has been shown that TGFP activity in NSCLC subtypes correlates with a clinical response to radiation. FIG. 5A provides representative images of adenocarcinoma (AD) and small cell lung carcinoma (SCC) human tumors stained with TGFβ and phospho-SMAD2 (a downstream signaling molecule of TGFβ). FIGS. 5B and 5C show that adenocarcinoma tumors express TGFβ at higher levels than SCC tumors. (See Du S, Quyang H, Pellicciotta I, Beheshti A, Lo C H, Parry R, and Barcellos-Hoff M H (2016)).

Biomarkers such as genomic biomarkers, immune cell markers, tumor cell markers, circulating markers, stem cell markers, and the like can be useful for predicting tumor response or sensitivity to radiotherapy. As such, these biomarkers may be expressed in radiation non-responsive or responsive cells and may be indicators of a clinical response to radiotherapy or a lack thereof.

### Example 2

### Combination Treatment Comprising an Immune Modulator and Radiation can Enhance Inhibition of Tumor Growth Compared to Monotherapy

In this study female B57/BL6 mice (n=5) were transplanted with MC38 colon carcinoma tumor pieces (2×2 mm). The tumors were exposed to gamma radiation (2 Gy) at day 8 post-transplantation. The mice were also admininistered an immune modulator, such as an anti-TIM4 antibody, an anti-MFG-E8 antibody, and an anti-M199 antibody at day 9 and day 11 post-transplantation. 2 mg/kg antibody was injected into each mouse. Tumor volumes were measured along three orthogonal axes (x, y, z) and tumor volume was calculated.

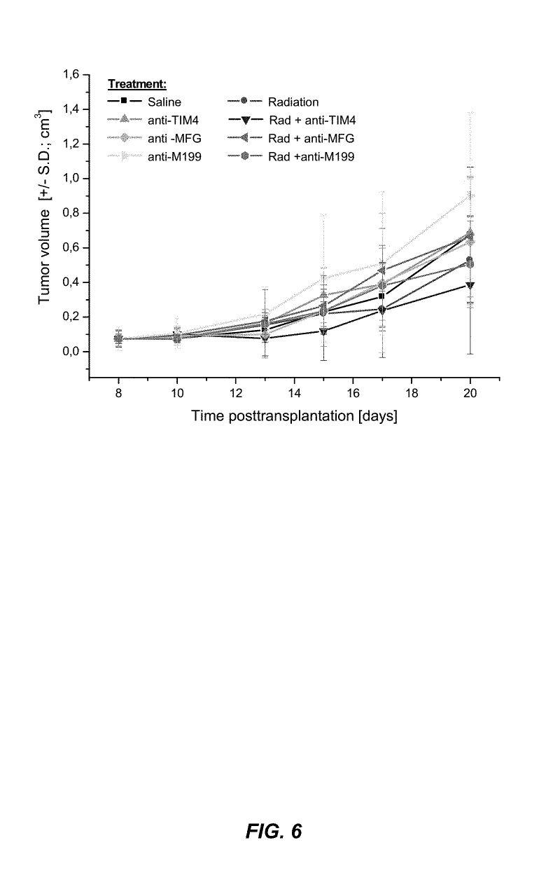

FIG. 6 shows that combination therapy of radiation and an anti-TIM4 antibody resulted in lower tumor growth compared to radiation therapy alone or anti-TIM4 antibody alone. In addition, combination therapy of radiation and an anti-M199 antibody also shows decreased tumor growth compared to anti-M199 antibody monotherapy. Similarly, anti-MFG-E8 antibody therapy in combination with radiation enhanced tumor growth inhibition compared to monotherapy. The results shows tumor growth inhibition in an immune competent animal model of cancer that has been administered a combination therapy.

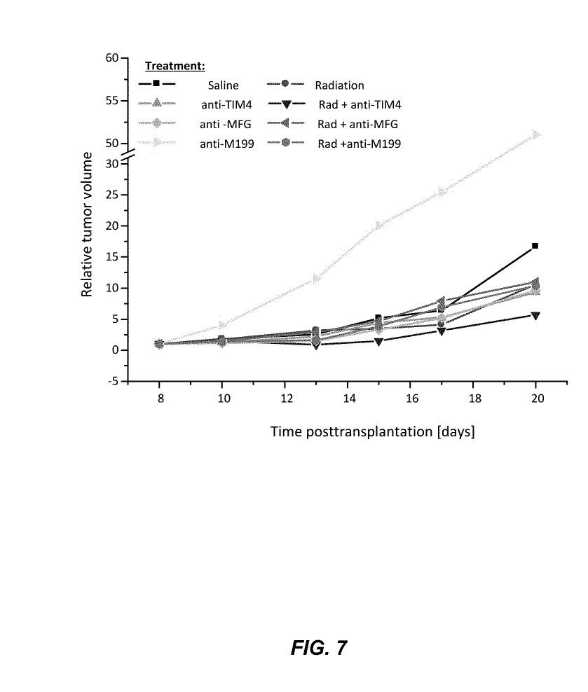

When the relative tumor volume was evaluated, combination therapy comprising radiation and either an anti-TIM4 antibody, anti-MFG-E8 antibody, or an anti-M199 antibody showed enhanced inhibition of tumor growth compared to treatment with an immune modulator alone (FIG. 7). The data shows that radiation in combination with immune modulator therapy can increase the anti-tumor response relative to radiation therapy alone.

### Example 3

### TIM-4 and MFGE-8 Protein Expression Levels in Human Tumor Samples

Material and methods: Formalin-fixed, paraffin-embedded tissue sections were de-paraffinized prior to staining with antibodies targeting either TIM-4 or MFGE-8. The staining was performed using two antigen retrieval methods: TIM-4—Target Retrieval Solution (Dako), Citrate buffer pH 6.1 at 97° C. for 20 minutes; MFGE-8—Target Retrieval Solution (Dako), Tris EDTA pH 9.0 at 97° C. for 20 minutes. Tissue sections were stained using a Dako Envision Flex Kit. Briefly, endogenous peroxides were blocked for 10 minutes with a peroxidase-blocking reagent. For mouse tumor tissues, slides were incubated with peroxidase blocking buffer for 1 hour. Mouse tumor tissue slides were rinsed in washing buffer and then incubated with Fc receptor blocker for 30 minutes. Mouse tissue sections were also incubated using mouse detective (Biocare) for 30 minutes. Tissue sections were incubated with the primary antibody targeting either TIM-4 or MFGE-8 for 30 minutes at RT for human tissues and overnight at 4° C. for mouse tissues. Mouse monoclonal antibody MFG-E8 ( 1/500 for human; Santa Cruz) and Rabbit polyclonal TIM-4 ( 1/500 for human, 1/400 for mouse; Abcam). Isotype controls and negative controls were run in parallel with respective primary antibodies to rule out any nonspecific staining. Tissues were incubated with the appropriate mouse, rabbit or mouse linker for 10 minutes, washed and then incubated in Dako Envision™+Dual Link System horse radish peroxidase (mouse and rabbit) for 30 minutes. Tissue section were stained for 10 minutes using a DAB chromogen mix and later counterstained with hematoxylin to visualize the nuclei.

Quantification of IHC Expression: The expression of each protein marker was assessed by its intensity and proportion following the methods given below: Briefly, intensity (abbreviated “Int”) is scored from 0 to 3 with 0=negative, 1=weak, 2=intermediate, and 3=strong. Proportion (abbreviated “Prop”) is scored from 0 to 4 with 0 through 5 corresponding to 0, 1-10, 21-50, 51-80, 81-100%, respectively. Total score (abbreviated “Tot”) is a multiplication of intensity and proportion and has values of 0-12.

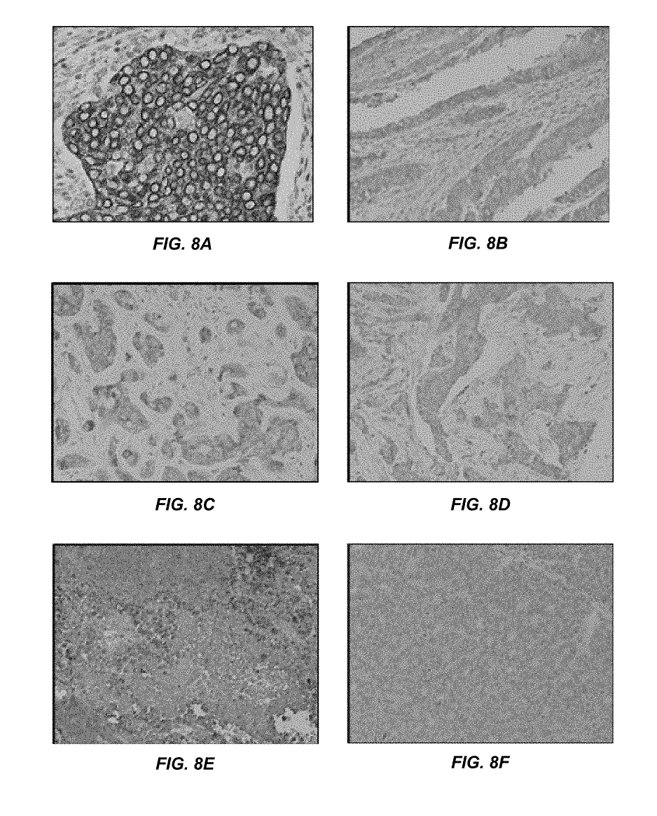

Results: TIM-4 expression was detected in human lung tumors, colon tumors, prostate tumors and breast tumors, and in a tumor bearing syngeneic mouse models, including MC-38 tumor bearing C57/BL6 model (FIG. 8A-8E).

Expression levels of TIM-4 were also evaluated in tumor tissue microarrays (BC041114, LUC481, Biomax, Inc.). TIM-4 expression was evaluated in 106 human lung tumor cases in total. Out of 90 lung tumors (BC041114), 10 cases showed strong staining, 50 cases showed moderate staining, and 30 cases weak staining. Out of 16 human lung tumor cases (LUC481), 4 lung tumor cases showed moderate staining, and 12 cases showed weak staining. TIM-4 expression was also evaluated in a human multi-organ tumor microarray (TMA2001, Biomax Inc.).

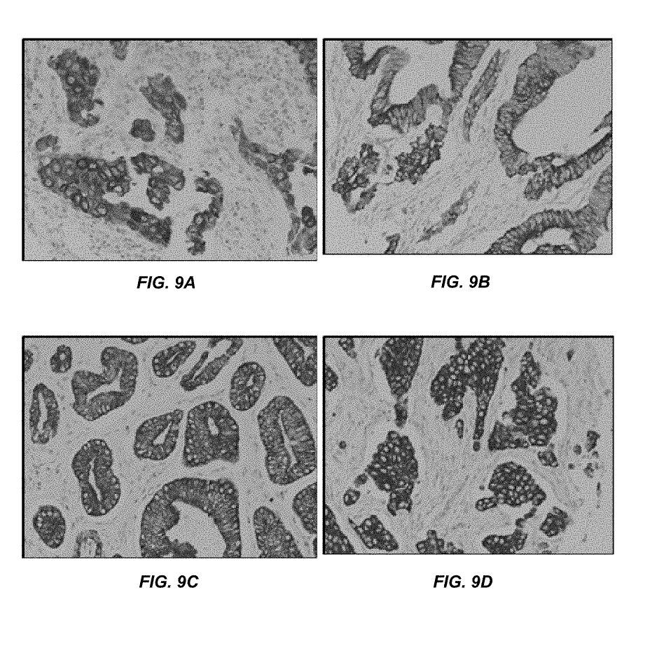

MFGE-8 expression was evaluated in a human multi-organ tumor microarray (TMA 2001, Biomax Inc.) and was detected in multiple tumors, including lung, colon, prostate and breast tumors (FIG. 9A-8D)

### Example 4

### Combination Treatment Comprising an Immune Modulator and Radiation can Inhibit Tumor Growth Compared to Monotherapy

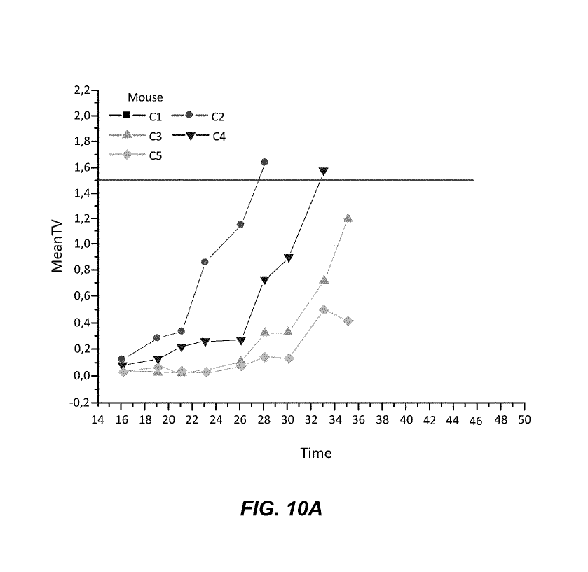

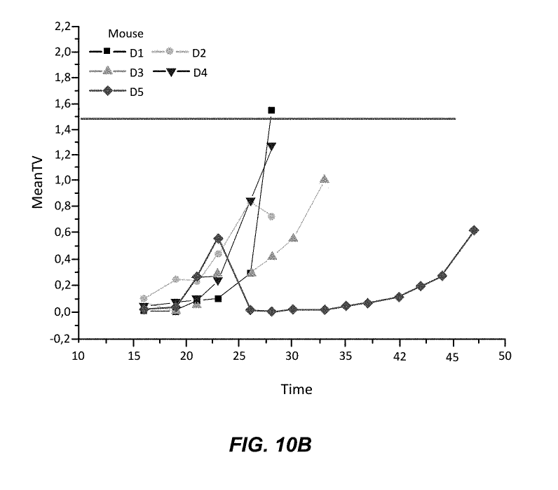

Tumor bearing animals (MC-38 bearing C57/BL6 mice) were treated with either anti-TIM-4 antibody alone (FIG. 10A) or anti-TIM-4 in combination with radiation (FIG. 10B). FIG. 10A shows MC-38 carcinoma bearing mice were treated with anti-TIM4 antibody (2mg/kg) on days 17.19,21,23. Tumor volumes of individual mice (C1-C5) were monitored over the course of the treatment. FIG. 10B shows MC-38 carcinoma bearing mice were treated with radiation (2 Gy) at day 16, followed by anti-TIM4 antibody administration (2 mg/kg) on days 17.19,21,23. Tumor volumes of individual mice (D1-D5) wer monitored over the course of the treatment. Tumor growth was monitored for up to 50 days. In some cases, as shown in FIG. 10B as an example, the tumor regressed after the initial tumor volume increased.

This example provides additional data showing that treatment of tumors with radiation in combination with an immune modulator can increase the anti-tumor response relative to immune modulator therapy alone.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

1. A method for treating a tumor in a subject with cancer comprising administering an effective amount of ionizing radiation and an immune modulator to the tumor.

2. The method of claim 1, wherein the immune modulator is selected from the group consisting of an inhibitor to an inhibitory checkpoint molecule, an activator of a stimulatory checkpoint molecule, a chemokine inhibitor, an inhibitor of macrophage migration inhibitory factor (MIF), a growth factor, a cytokine, an interleukin, an interferon, an antibody that binds to an immune system cell, a cellular immune modulator, a vaccine, an oncolytic virus, and any combination thereof.

3. The method of claim 2, wherein the inhibitor to the inhibitory checkpoint molecule is a small molecule drug, or an antibody or a fragment thereof that specifically binds to the inhibitory checkpoint molecule and inhibits its activity, wherein the inhibitory checkpoint molecule is selected from the group consisting of PD-1, PD-L1, PD-L2, CTLA-4, BTLA, A2aR, B7-H2, B7-H3, B7-H4, B7-H6, CD47, CD48, CD160, CD244 (2B4), CHK1, CHK2, CGEN-15049, ILT-2, ILT-4, LAG-3, VISTA, gp49B, PIR-B, TIGIT, TIM1, TIM2, TIM3, TIM4, and KIR, and ligands thereof.

4. The method of claim 2, wherein the activator of the stimulatory checkpoint molecule is a small molecule drug, polypeptide-based activator, or polynucleotide-based activator that specifically binds to the stimulatory checkpoint molecule and increases its activity, wherein the stimulatory checkpoint molecule is selected from the group consisting of B7-1 (CD80), B7-2 (CD86), 4-IBB (CD137), OX40 (CD134), HVEM, inducible costimulator (ICOS), glucocorticoid-induced tumor necrosis factor receptor (GITR), CD27, CD28, CD40, and ligands thereof.

5. The method of claim 2, wherein the chemokine inhibitor is (i) a small molecule drug, or antibody or fragment thereof that specifically binds to the chemokine and inhibits chemokine activity, wherein the chemokine is selected from the group consisting of CCL2, CCL3, CCL4, CCL5, CCL7, CCL8, CCL11, CCL12, CCL13, CCL14, CCL15, CCL16, CCL17, CCL18, CCL19, CCL20, CCL21, CCL22, CCL23, CCL24, CCL5, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL5, and CXCL16 or (ii) a small molecule drug, or antibody or fragment thereof that specifically binds to a chemokine receptor and inhibits chemokine activity, wherein the chemokine receptor is selected from the group consisting of CCR1, CCR2, CCR3, CCR, 4, CCR5, CCR6, CCR7, CCR8, CCR9, CCR10, CXCR1, CXCR2, CXCR3, CXCR4, CXCRS, CXCR6, and CXCR7.

6. (canceled)

7. The method of claim 2, wherein the inhibitor of MIF is a small molecule drug, or antibody or fragment thereof that specifically binds to MIF and inhibits MIF activity.

8. The method of claim 1, further comprising:
(a) detecting an expression level of one or more biomarkers in a tumor sample from the subject, wherein the one or more biomarkers are selected from the group consisting of an immune cell marker(s), tumor cell marker(s), circulating marker(s), and any combination thereof;
(b) comparing the expression level of the one or more biomarkers to the expression level of the one or more biomarkers in a normal tissue sample; and
(c) treating the tumor with ionizing radiation and an immune modulator if the expression level of the one or more biomarkers is modified compared to the expression level in the normal tissue sample,.
wherein the expression level of the one or more biomarkers is modified if the expression level of at least one of the biomarkers is increased, or the expression level of at least one of the biomarkers is decreased, or the expression level of at least one of the biomarkers is increased and the expression level of at least one of the biomarkers is decreased compared to the expression level in a normal tissue sample.

9. (canceled)

10. The method of claim 8, wherein the tumor sample is a biopsy comprising tumor cells, or
the normal tissue sample comprises non-tumor cells from the same tissue type as the tumor.

11. The method of claim 8, wherein the immune cell biomarker(s) or the tumor cell biomarker(s) or the circulating biomarker(s) is a polynucleotide or a protein, or
the detecting is performed by using an assay selected from the group consisting of immunohistochemistry, ELISA, Western analysis, HPLC, proteomics, PCR, RT-PCR, Northern analysis, and a microarray.

12. The method of claim 8, wherein the biomarker is CD44, MFG-E8, CD68, TGFβ, or a TGFβ-pathway related biomarker.

13. (canceled)

14. (canceled)

15. The method of claim 8, wherein the expression level of the one or more biomarkers is ranked or weighted.

16. The method of claim 8, further comprising performing functional imaging of the tumor prior to administering the ionizing radiation and the immune modulator.

17. The method of claim 8, wherein the ionizing radiation and/or the immune modulator is administered at a higher dose compared to a standard treatment protocol if the expression level of the one or more biomarkers in the tumor sample is modified compared to the expression level in the normal tissue sample.

18. The method of claim 17, wherein the expression level of CD44 is increased and the expression level of MFG-E8 is decreased compared to the expression level in the normal tissue sample, or
the expression level of CD68 is increased compared to the expression level in the normal tissue sample.

19. (canceled)

20. The method of claim 8, wherein the ionizing radiation is administered as a hypofractionated or a hyperfractionated radiation treatment if the expression level of the one or more biomarkers in the tumor sample is modified compared to the expression level in the normal tissue sample, or
the ionizing radiation and the immune modulator are administered concomitantly or sequentially.

21. (canceled)

22. (canceled)

23. (canceled)

24. A method of treating a tumor in a subject with cancer, the method comprising:
(a) determining an expression level of one or more biomarkers in a tumor sample from the subject, wherein the one or more biomarkers are selected from the group consisting of an immune cell marker(s), tumor cell marker(s), circulating marker(s), and any combination thereof;
(b) comparing the expression level of the one or more biomarkers to an expression level of the one or more biomarkers in a normal tissue sample; and
(c) administering to the tumor in the subject a treatment comprising ionizing radiation and an immune modulator if the expression level of the one or more biomarkers in the tumor sample is modified compared to the expression level in the normal tissue sample
wherein the expression level of the one or more biomarkers is modified if the expression level of at least one of the biomarkers is increased, or the expression level of at least one of the biomarkers is decreased, or the expression level of at least one of the biomarkers is increased and the expression level of at least one of the biomarkers is decreased compared to the expression level in a normal tissue sample.

25. (canceled)

26. (canceled)

27. The method of claim 24, wherein administering ionizing radiation comprises contacting the tumor with a radiosensitizer.

28-50. (canceled)

51. A method of selecting a treatment for a subject with cancer, the method comprising:
(a) determining an expression level of one or more biomarkers in a tumor sample from the subject, wherein the one or more biomarkers are selected from the group consisting of an immune cell marker(s), tumor cell marker(s), circulating marker(s), and any combination thereof;
(b) comparing the expression level of the one or more biomarkers to an expression level of the one or more biomarkers in a normal tissue sample; and
(c) selecting a treatment comprising ionizing radiation and an immune modulator if the expression level of the one or more biomarkers in the tumor sample is modified compared to the expression level in the normal tissue sample.

52.-66. (canceled)

67. The method of claim 1, further comprising administering a radiosensitizer to the tumor.
